(19) United States
(12) Patent Application Publication
(10) Pub. No.: US 2013/0190565 A1
(43) Pub. Date: Jul. 25, 2013

(54) SYSTEM, METHOD AND APPARATUS FOR OPTICAL IMAGING OF LUMINAL ORGANS, AND FOR CENTERING WITHIN AND CONTACTING A LUMINAL ORGAN

(71) Applicant: The General Hospital Corporation, Boston, MA (US)

(72) Inventors: Michalina Jozefina Gora, Somerville, MA (US); Gregory Dierksen, Brighton, MA (US); Brett Eugene Bouma, Quincy, MA (US); Guillermo J. Tearney, Cambridge, MA (US)

(73) Assignee: The General Hospital Corporation, Boston, MA (US)

(21) Appl. No.: 13/744,935

(22) Filed: Jan. 18, 2013

Related U.S. Application Data
(60) Provisional application No. 61/589,083, filed on Jan. 20, 2012.

(51) Int. Cl. A61B 1/00; A61B 18/18

(52) U.S. Cl. CPC A61B 1/00082; A61B 18/18
USPC 600/116; 606/33

(57) ABSTRACT

Exemplary apparatus and method can be provided for obtaining data for at least one portion within at least one luminal or hollow sample. It is possible, e.g., using a first optical arrangement, to transceive at least one electromagnetic radiation to and from the portion(s). Further, it is possible, e.g., to actuate an expandable second basket arrangement so as to position the first arrangement at a predetermined location within the luminal or hollow sample(s). The second arrangement can have at least one prong or strip that has a flat shape on at least one side thereof.

### CROSS-REFERENCE TO RELATED APPLICATION(S)

This application is based upon and claims the benefit of priority from U.S. Patent Application Ser. No. 61/589,083 filed on Jan. 20, 2012, the entire disclosure of which is incorporated herein by reference.

### FIELD OF THE DISCLOSURE

Exemplary embodiments of the present disclosure relate to system, method and apparatus for optical imaging of luminal organs, and for centering and contacting a luminal organ.

### BACKGROUND INFORMATION

An optical imaging device has become an important tool to assess and diagnose diseases arising from luminal organs. Imaging methods/procedures including optical coherence tomography (OCT) and optical frequency domain imaging (OFDI) are two exemplary gastrointestinal tissue imaging methods. Other exemplary methods/procedures include confocal microscopy and spectrally-encoded confocal microscopy (SECM). OCT and OFDI procedures can acquire back-scattered light that comes from the refractive index mismatch of cellular and sub-cellular components, thereby facilitating the generation of images of at least one tissue microstructure in vivo. Comprehensive imaging, achieved by the helical pullback scanning of the imaging optics, can facilitate such microscopic imaging information to be obtained from entire sections of one or more luminal organs. Based on this microstructural information, diseases from the luminal organs, such as esophagus, colon, vessels, ducts, and so on, can be identified and detected in the early stages.

Optical tomography methods/procedures, including OCT and OFDI, can have a limited imaging depth range, e.g., from a few hundred micrometers to several millimeters. To obtain sufficient image contrast and resolution, the tissue should be located within the optical imaging range. In case of many luminal organs, which can have relatively large diameter (e.g., 5-8 cm for human colon, 2.5 cm for human esophagus, etc.), one preferable way to obtain images of the entire organ can be a centration of the imaging probe within the lumen.

One possibility for such centration can be to utilize a balloon catheter. After the placement of the catheter, the balloon can be inflated, thus resulting in the centration of the imaging optics. This procedure facilitates the imaging catheter to obtain the images from the entire epithelial tissues of the luminal organs. Since balloon can be inflated and deflated, and the balloon-catheter can be used as a standalone device or with the endoscope through the accessory channel.

However, the luminal organs can have complex structures thus causing the bending of the catheter and the decentering of the optical probe. This can cause the suboptimal imaging of the luminal organs. Some areas of the luminal organ may not be appropriately imaged when the decentering is greater than the imaging probe's imaging depth. Furthermore, due to the movement by the patient, which includes breathing and heart beating, the bending issues and the associated decentering can occur frequently in a clinical setting, thus causing incomplete image acquisition.

Currently, a placement of the OFDI balloon catheter utilizes a sedated upper endoscopy procedure. Unfortunately, upper endoscopy can be a costly procedure. An important contributor to the high cost of endoscopy is the preference of sedation, which can force the procedure to be conducted in a specialized environment, with continuous cardiopulmonary monitoring and nursing support. Patient cost can be an additional factor, as sedation results in prolonged recovery times and loss of productivity.

In general when passing a catheter, transnasal access can be better tolerated than the transoral approach because of a more vigorous gag reflex encountered in unsedated transoral procedures. Standard transnasal procedures, such as nasogastric tube (NG tube) insertion, can be conducted in millions of patients annually, with few (if any) major complications. Unsedated, transnasal balloon dilation can be conducted in the outpatient setting, without complication, and is well tolerated. (See Rees C J, “In-office unsedated transnasal balloon dilation of the esophagus and trachea. Current opinion in otolaryngology & head and neck surgery”, 2007; 15(6):401-4). Because the diameter of the balloon catheter is small enough to be threaded into the standard nasogastric tube, it can be also used for esophagus imaging procedures, without sedation.

Another form of the OFDI catheter facilitating unsedated procedure is a capsule that can be swallowed. The endoscopic capsule endoscopy (ECE) can be easier to administer than transnasal endoscopy and, since swallowing a capsule is familiar to patients, and it can be better tolerated than transnasal procedures. Conventional capsule endoscopy procedures likely have a lack of control of the capsule at the GEJ, however, thus possibly resulting in few viable images obtained at the critical region of the esophagus. Due to the decreased diagnostic accuracy and the high cost of the single-use, disposable capsule (e.g., about $450), the cost-effectiveness analyses for BE screening with capsule endoscopy have not demonstrated a benefit over conventional endoscopy. Another procedure, i.e., string capsule endoscopy (SCE) can be used, and which tethers the capsule with a string to enable strict control of the pill camera's location and repeated visualization of the GEJ. (See Weston A P, “String capsule endoscopy: a viable method for screening for Barrett's esophagus”, Gastrointestinal endoscopy. 2008; 68(1):32-4). A recent study in 100 patients with SCE showed that this technique is well tolerated and has a comparable diagnostic performance to that of upper endoscopy. For example, the SCE capsules can be retrieved, sterilized, and reused, thereby significantly decreasing the cost of the capsule endoscopy. Nonetheless, the SCE procedures are likely subject to the same diagnostic accuracy limitations as endoscopy, however.

An important characteristic of balloon catheters is the influence of the balloon on the tissue. In general, the centration balloon compresses imaged tissue, which can influence the diagnostic accuracy. The diagnostic process/procedure can be based on the structural differences that are characteristic for healthy and diseased tissues. Surface topology can be helpful in the analysis of the results, e.g., finger like projection in the epithelium is a typical feature for Barrett's esophagus. Additionally, a validation of the OFDI catheter imaging method/procedure can be performed by comparing the biopsy taken from the imaged region. It may be difficult to mimic exactly the same pressure conditions for the histology specimens.

Thus, it may be beneficial to address and/or overcome at least some of the deficiencies of the prior approaches, procedures and/or systems that have been described herein above.

### SUMMARY OF EXEMPLARY EMBODIMENTS

It is therefore one of the objects of the present disclosure to reduce or address the deficiencies and/or limitations of such prior art approaches, procedures, methods and systems. Such objects can be effectuated using exemplary embodiments of system, method and apparatus for optical imaging of luminal organs, and for centering and contacting a luminal organ.

According to an exemplary embodiment of the present disclosure, a basket catheter/endoscope device can be provided for optical imaging of luminal organs that can include an arrangement for centering and contact with the luminal organ. It is possible to utilize any of the following optical imaging technology, such as, OCT, time domain (TD)-OCT, spectral domain (SD)-OCT, OFDI, SECM or fluorescence confocal microscopy. It should be understood that other imaging technologies can be utilized with the exemplary embodiments of the present disclosure.

In one exemplary embodiment of the present disclosure, exemplary apparatus and method can be provided for obtaining data for at least one portion within at least one luminal or hollow sample. It is possible, e.g., using a first optical arrangement, to transceive at least one electromagnetic radiation to and from the portion(s). Further, it is possible, e.g., to actuate an expandable second basket arrangement so as to position the first arrangement at a predetermined location within the luminal or hollow sample(s). The second arrangement can have at least one prong or strip that has a flat shape on at least one side thereof.

For example, the prong(s) or strip(s) can include a plurality of prongs, each of which having a flat shape and/or at least one of which being at least partially transparent. A computer arrangement can be provided which receives information regarding the sample after the at least one prong or strip stabilizes, configured to center the sample with respect to the apparatus, and configured to generate at least one image of the portion within the sample(s). According to another exemplary embodiment of the present disclosure, the prong(s) or strip(s) can have at least two sides parallel to one another, and a flat shape on the sides (e.g., with such side contacting the sample).

In yet another exemplary embodiment of the present disclosure, a computer arrangement can be provided which receives information regarding a shape or a characteristic of the second arrangement, and configured to compensate for at least one aberration within the prong(s) or strip(s). The apparatus can be structured and sized to be insertable via a mouth and/or a nose of a patient. The second arrangement can includes at least one section which facilitates a guiding arrangement to be inserted there through. A further arrangement can be provided which can be configured to measure a pressure within the portion(s).

The information received by the computer arrangement can include a position and/or an orientation of the first arrangement with respect to the sample(s). The electromagnetic radiation(s) can be provided at one or more wavelengths in a visible range. The first arrangement can include a section which can direct the electromagnetic radiation(s) toward the portion(s), and can obtain the data. The first optical arrangement can be configured to transceive at least one first electromagnetic radiation to and from the portion(s), and transmit at least one second electromagnetic radiation so as to ablate, thermally damage or produce a structural change of or in the portion(s).

The information received by the computer arrangement can be interferometric data associated with the sample(s). The interferometric data can be spectral-domain optical coherence tomography data, optical frequency domain imaging data and/or confocal data.

According to still another exemplary embodiment of the present disclosure, a further apparatus can be provided which can be configured to receive and record the information and a position and a rotational angle of the first arrangement with respect to the sample(s). For example, the further arrangement can include a scanning arrangement, and the further arrangement can detect the position and the rotation angle by digital counting of encoder signals obtained from the scanning arrangement during at least one scan of the sample(s). An additional arrangement can also be provided which can be configured to receive the position and the rotational angle, and generate at least one image associated with the portion(s) using the position and the rotational angle.

In a further exemplary embodiment of the present disclosure, a processing arrangement can be provided which can be controlled to receive a plurality of images of the sample(s) during at least two axial translations of the first arrangement with respect to the sample(s). For example, each of the axial translations can be provide at a rotational angle. At least one portion of the apparatus can be coated with an anesthetic substance.

These and other objects, features and advantages of the present disclosure will become apparent upon reading the following detailed description of exemplary embodiments of the present disclosure, when taken in conjunction with the appended drawings and claims provided herewith.

### BRIEF DESCRIPTION OF THE DRAWINGS

Further objects, features and advantages of the present disclosure will become apparent from the following detailed description taken in conjunction with the accompanying drawings showing illustrative embodiments of the present invention, in which:

FIG. 1A is a front cross-sectional view of a basket catheter/endoscope device/system according to an exemplary embodiment of the present disclosure in an exemplary closed configuration;

FIG. 1B is a front cross-sectional view of the basket catheter/endoscope device/system of FIG. 1A in an exemplary deployed (or opened)configuration;

FIG. 2 is a side cross-sectional view of the basket catheter/endoscope device/system of FIG. 1A in the closed configuration;

FIG. 3 is a side cross-sectional view of the basket catheter/endoscope device/system of FIG. 1A in the deployed (or opened) configuration; and

FIGS. 4A and 4B are exemplary endoscopic photographs from imaging of swine esophagus in vivo using the exemplary catheter/endoscope device/system, in a closed configuration of the basket, and in a deployed configuration, respectively, according to the exemplary embodiment of the present disclosure;

FIG. 4C is an exemplary two-dimensional (2D) cross-sectional frame from the three-dimensional (3D) OFDI data set obtained after a deployment of the exemplary catheter/system/device according to one exemplary embodiment of the present disclosure using, e.g., four expanding elements; and

FIG. 4D shows an exemplary 2D cross-sectional frame from the 3D OFDI data set obtained after deployment of the exemplary catheter/system/device according to another exemplary embodiment of the present disclosure with, e.g., 6 softer prongs.

Throughout the drawings, the same reference numerals and characters, if any and unless otherwise stated, are used to denote like features, elements, components, or portions of the illustrated embodiments. Moreover, while the subject disclosure will now be described in detail with reference to the drawings, it is done so in connection with the illustrative embodiments. It is intended that changes and modifications can be made to the described exemplary embodiments without departing from the true scope and spirit of the subject disclosure and appended claims provided herewith.

### DETAILED DESCRIPTION OF EXEMPLARY EMBODIMENTS

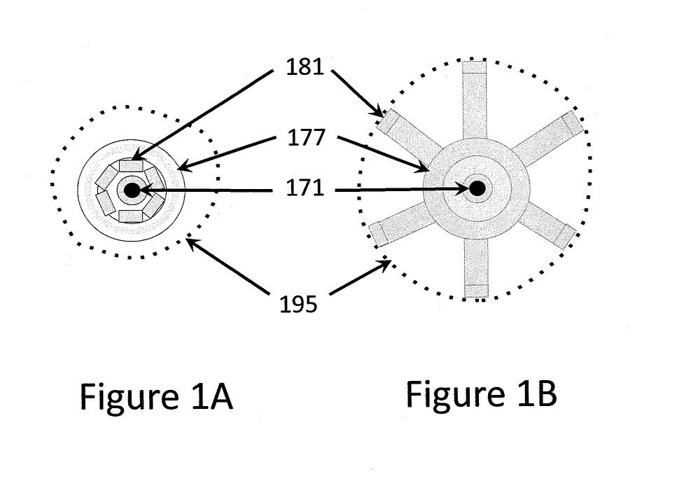

FIGS. 1A, 1B, 2 and 3 depict various cross-sectional views of a basket catheter/endoscope device/system according to an exemplary embodiment of the present disclosure in different deployment configurations. For example, the exemplary catheter/device/system can include an optical probe 171 that can focus and redirect an optical beam 199 into a tissue 195. The exemplary optical probe 171 can be enclosed, at least partially, in an inner sheath 175 that can protects such probe 171 from damage and/or contact with the environment. The exemplary optical probe 171 can rotate and/or translate within the inner sheath 175, and comprises at least one of optical fibers, driveshaft, lens, grating and/or redirecting optics.

The inner sheath 175 can be placed in a flexible outer tube 177 that can be connected to a basket 178 via a small tube 180. This tube 180 (e.g., which can be referred to as a loose junction tube 180) can have an inner diameter large enough to facilitate sliding of the inner sheath 175 inside thereof. For example, the basket 178 can be made of at least one (and possible more, such as four to 8) transparent plastic prong 181, and stiff enough for opening of the esophagus or other luminal organ lumen. The plastic prongs 181, in one exemplary embodiment, can be flat on one side, and configured to be in stable contact with the underlying tissue 195 when the basket 178 is deployed (FIG. 1). The basket 178 and the inner sheath 175 can be connected to the soft tip 183 using a tight tube 185 (which can be referred to as a tight junction tube 185). Both the loose junction tube 180 and the tight junction tube 185 can overlap with the basket 178 so as to reduce and/or prevent the prongs 181 from bending in a wrong direction.

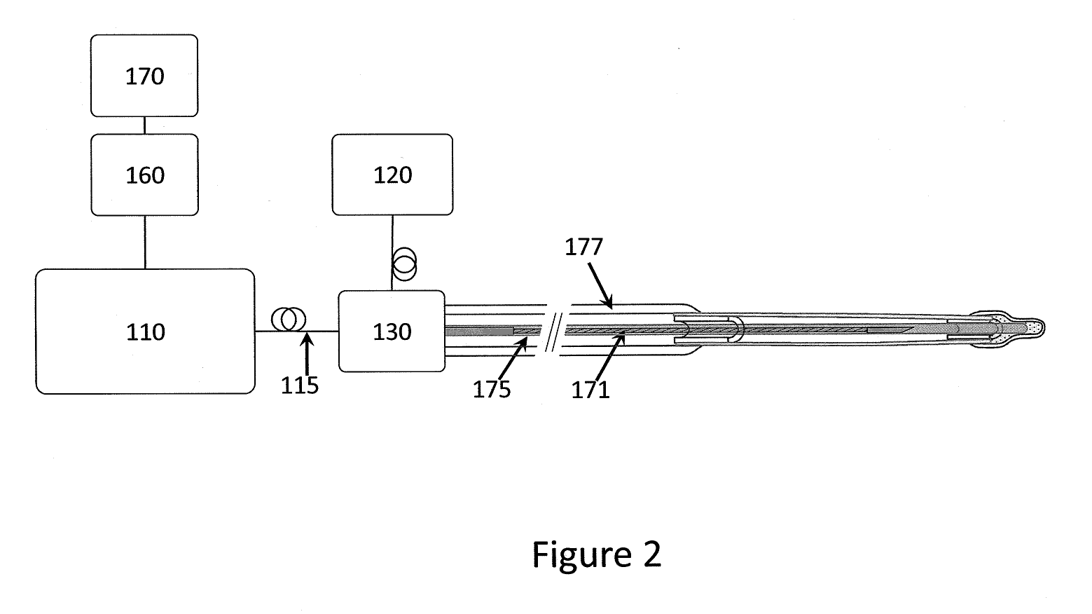

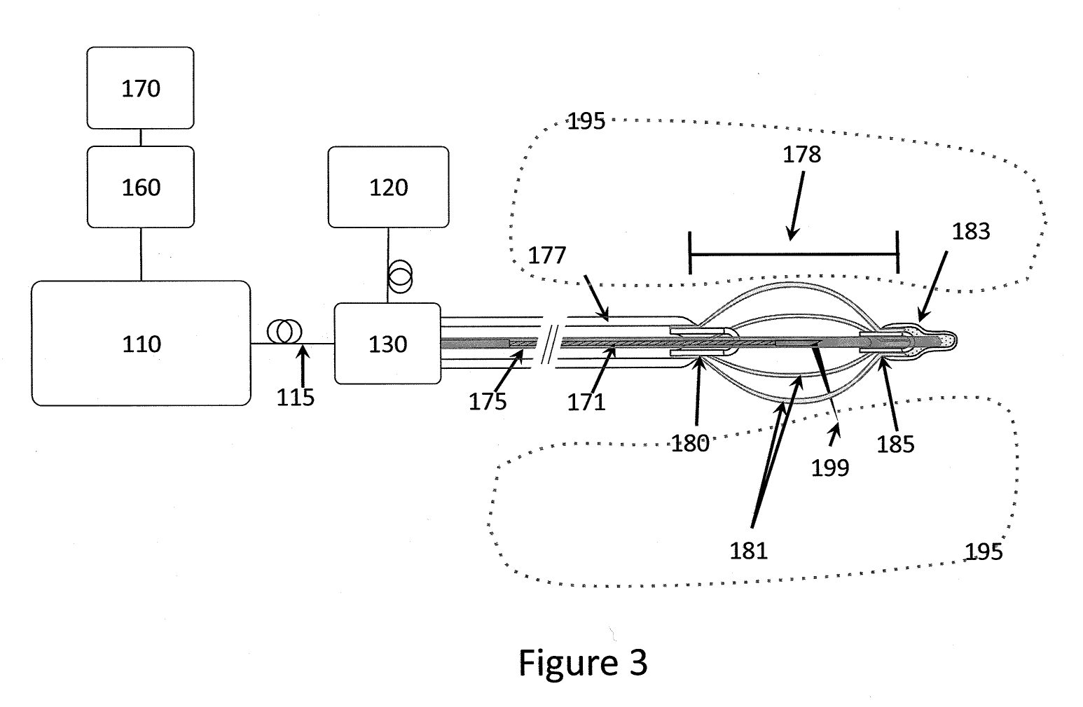

When the exemplary catheter/device/system is placed in the esophagus or in other luminal organ, the basket 178 can be closed (as shown in FIG. 2). The basket 178 can be deployed by pulling on the inner sheath 175, which can causes bending of the prongs 181. Indeed, FIG. 2 depicts the basket 178 in its exemplary undeployed state, and FIG. 3 depicts the basket 178 in its exemplary deployed state. The basket 178 can be inserted through a natural orifice (such as, e.g., the nose or mouth) to be positioned within a luminal organ such as the esophagus or stomach so that the exemplary catheter/device/system can obtain imaging of a portion of the anatomical structure.

The exemplary optical probe/catheter/device can be connected to an additional light source 120 for laser marking through an optical rotary junction 130 operatively connected to an interferometric apparatus that can include a reference 110. Rotary junction 130 provides helical scanning of the optical probe 171 and provides information necessary for data reconstruction. The light (or any other electro-magnetic radiation) returned from the sample can be detected using a detector apparatus 160, and processed by a CPU 170 (or another computing arrangement) to obtain an optical image.

### Exemplary Design Specifications

Exemplary Dimensions

In one exemplary embodiment of the present disclosure, the length and diameter of the device/catheter/probe and/or its components can be selected so as to facilitate the insertion thereof through a natural orifice and directed to an area of interest on the luminal anatomic structure. For example, for a transnasal application, an exemplary maximum outer diameter of the device when the basket is not deployed can be approximately 16 Fr=5.3 mm.

Exemplary Structural Configuration

In one exemplary embodiment of the present disclosure, as indicated herein, the device/catheter/probe can comprise an inner protective sheath that houses an optical probe and a basket mechanism for positioning the optical probe at a predetermined location in the lumen of a luminal organ. The optical probe can be configured to focus the light into the tissue of the luminal anatomic structure and move to scan the focused beam on or within the luminal organ. In another exemplary embodiment, the basket mechanism can be configured to center the optical probe within the luminal organ. The basket mechanism can contain one prong or a plurality of prong elements. In a particular exemplary embodiment of the present disclosure, the prongs can be flat, and can have an aspect ratio >1 so that they maintain stable contact with the tissue when deployed. In yet another exemplary embodiment, the prongs can be optical transparent for visible or NIR electromagnetic radiation.

Exemplary Flexibility

According to one exemplary embodiment of the present disclosure, the assembled catheter/device/probe can have a bend radius of approximately 80 mm diameter, and can be configured to pass through the nose and transnasal tract so that it may enter the esophagus, stomach or intestine.

Exemplary Probe Centration

In an exemplary embodiment of the present disclosure, the basket can open to a diameter ranging from about 15 mm to 25 mm. In yet another exemplary embodiment, the diameter can be fixed or adjustable. In still another exemplary embodiment, the basket elements can be individually adjusted to position the optical probe at an arbitrary location in the lumen of the luminal organ with respect to the wall of the luminal organ.

Exemplary Imaging Window

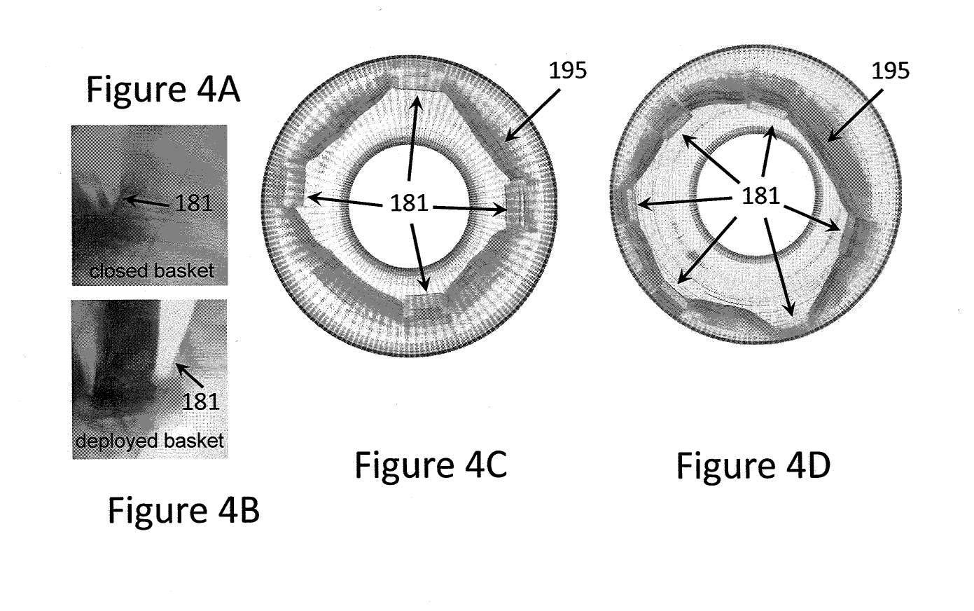

According to an exemplary embodiment of the present disclosure, the imaging window has a length of 6 cm (length in which optical beam can be pulled back and image without any obstruction, except centering elements). In another one exemplary embodiment of the present disclosure, the length over which imaging can be conducted is 3 cm. In yet another one exemplary embodiment, the imaging window length can be on the order of 1 cm. In a further exemplary embodiment, the basket material can be transparent, so as to facilitate imaging there through (shown in FIG. 4).

Indeed, FIG. 4A-4D illustrates exemplary results from imaging of swine esophagus in vivo. In particular, FIG. 4A illustrates an exemplary endoscopy photo of a closed catheter, and FIG. 4B illustrates an exemplary endoscopy photo of a deployed catheter, both with, e.g., four (4) stiff expanding elements 181 with respect to the tissue 195. FIG. 4C illustrates an exemplary 2D cross-sectional frame from the 3D OFDI data set obtained after a deployment of the catheter using four expanding elements 181, and FIG. 4D shows an exemplary 2D cross-sectional frame from the 3D OFDI data set obtained after deployment of the catheter with, e.g., 6 softer prongs. The expanding elements 181 can be, e.g., transparent so as to not obscure the tissue 195.

Exemplary computer processing methods can correct for the refractive index change in optical path length induced by the basket elements.

Exemplary Catheter Placement

In an exemplary embodiment of the present disclosure, the outer tube distal tip can be soft and rounded for easy placement. In another exemplary embodiment of the present disclosure, the exemplary catheter/system/device can be used for guiding the arrangement over a guidewire. For example, the soft outer tube can be connected to the basket or being loose and covering the basket section during placement and than be retracted to exposed the basket. The exemplary placement in the esophagus can be performed based on real time images acquired with the catheter with closed basket or by using additional pressure sensor in the close vicinity of the basket section of the catheter/system/device.

Exemplary Basket

According to an exemplary embodiment of the present disclosure, it is possible to have one expanding element or a plurality of expanding elements. The expanding element(s) can be, e.g., flat wires or narrow width plastic prongs. In one further exemplary embodiment, the expanding element(s) can be small in order to decrease contact with a tissue, but still be able to open the lumen and kept it in semi-circular shape.

### Exemplary Result

The catheter/system/device according to an exemplary embodiment(s) of the present disclosure was tested with a swine model in vivo. Prototype basket catheters having different material properties and numbers of expanding elements were tested. After a placement in the lower esophagus with assistance of endoscopy, the exemplary catheter was opened and a 3D OFDI dataset over a 3 cm pullback was collected (as shown in FIGS. 4A-4D). During the procedure, it was possible to adjust the diameter of the basket to and to open esophagus.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements and methods which, although not explicitly shown or described herein, embody the principles of the disclosure and are thus within the spirit and scope of the present disclosure. In addition, to the extent that the prior art knowledge has not been explicitly incorporated by reference herein above, it is explicitly being incorporated herein in its entirety. Further, the exemplary embodiments described herein can operate together with one another and interchangeably therewith. All publications referenced herein above are incorporated herein by reference in their entireties.

What is claimed is:

1. An apparatus for obtaining data for at least one portion within at least one luminal or hollow sample, comprising:
a first optical arrangement configured to transceive at least one electromagnetic radiation to and from the at least one portion; and
an expandable second basket arrangement configured to be actuated so as to position the first arrangement at a predetermined location within the at least one luminal or hollow sample, wherein the second arrangement-has at least one prong or strip that has a flat shape on at least one side thereof.

2. The apparatus according to claim 1, wherein the at least one prong or strip includes a plurality of prongs, each of which having a flat shape.

3. The apparatus according to claim 1, wherein the at least one prong or strip includes a plurality of prongs, at least one of which being at least partially transparent.

4. The apparatus according to claim 1, further comprising a computer arrangement which receives information regarding the sample after the at least one prong or strip stabilizes, configured to center the sample with respect to the apparatus, and configured to generate at least one image of the at least one portion of the at least one sample.

5. The apparatus according to claim 1, wherein the at least one prong or strip has at least two sides parallel to one another, and having a flat shape on the sides.

6. The apparatus according to claim 1, wherein the at least one side contacts the sample.

7. The apparatus according to claim 1, further comprising a computer arrangement which receives information regarding a shape or a characteristic of the second arrangement, and compensated for at least one aberration within the at least one prong or strip.

8. The apparatus according to claim 1, wherein the apparatus is structured and sized to be insertable via at least one of a mouth or a nose of a patient.

9. The apparatus according to claim 1, wherein the second arrangement includes at least one section which facilitates a guiding arrangement to be inserted there through.

10. The apparatus according to claim 1, further comprising a further arrangement which is configured to measure a pressure within the at least one portion.

11. The apparatus according to claim 4, wherein the information includes at least one of a position or an orientation of the first arrangement with respect to the at least one luminal or hollow sample.

12. The apparatus according to claim 4, wherein the at least one electromagnetic radiation is provided at one or more wavelengths in a visible range.

13. The apparatus according to claim 4, wherein the first arrangement includes a section which directs the at least one electromagnetic radiation toward the at least one portion, and obtains the data.

14. The apparatus according to claim 1, wherein the first optical arrangement is configured to transceive at least one first electromagnetic radiation to and from the at least one portion, and transmit at least one second electromagnetic radiation so as to ablate, thermally damage or produce a structural change of or in the at least one portion.

15. The apparatus according to claim 1, further comprising a further apparatus which is configured to receive and record the information and a position and a rotational angle of the first arrangement with respect to the at least one sample.

16. The apparatus according to claim 15, wherein the further arrangement includes a scanning arrangement, and wherein the further arrangement detects the position and the rotation angle by digital counting of encoder signals obtained from the scanning arrangement during at least one scan of the at least one sample.

17. The apparatus according to claim 15, further comprising an additional arrangement which is configured to receive the position and the rotational angle, and generate at least one image associated with the at least one portion using the position and the rotational angle.

19. The apparatus according to claim 1, further comprising a processing arrangement which is capable of being controlled to receive a plurality of images of the at least one sample during at least two axial translations of the first arrangement with respect to the at least one sample, wherein each of the axial translations is provide at a rotational angle.

20. The apparatus according to claim 4, wherein the information are interferometric data associated with the at least one sample.

21. The apparatus according to claim 20, wherein the interferometric data is at least one of spectral-domain optical coherence tomography data, optical frequency domain imaging data or confocal data.

22. The apparatus according to claim 1, wherein at least one portion of the apparatus is coated with an anesthetic substance.

23. A method for obtaining data for at least one portion within at least one luminal or hollow sample in an unsedated patient, comprising:
transceiving at least one electromagnetic radiation to and from the at least one portion using a first optical arrangement; and
actuating an expandable second arrangement so as to position the first arrangement at a predetermined location within the at least one luminal or hollow sample, wherein the second arrangement has at least one prong or strip that has a flat shape on at least one side thereof.
